(19) United States
(12) Patent Application Publication
(10) Pub. No.: US 2023/0174398 A1
(43) Pub. Date: Jun. 8, 2023

(54) FLUORESCENCE DETECTION OF SULFITE IN WATER TREATMENT APPLICATIONS

(71) Applicant: CHEMTREAT, INC., Glen Allen, VA (US)

(72) Inventors: Megan PETTYGROVE, Quinton, VA (US); Robert JEFFERS, Glen Allen, VA (US); Kevin BOUDREAUX, Sioux Falls, SD (US); Santanu BANERJEE, Glen Allen, VA (US)

(73) Assignee: CHEMTREAT, INC., Glen Allen, VA (US)

(21) Appl. No.: 18/076,964

(22) Filed: Dec. 7, 2022

Related U.S. Application Data
(60) Provisional application No. 63/286,791, filed on Dec. 7, 2021.

(51) Int. Cl. C02F 1/68; G01N 21/64; G01N 33/18

(52) U.S. Cl. CPC C02F 1/68; G01N 21/643; G01N 33/182; G01N 2021/6439

(57) ABSTRACT

The amount of sulfite in water can be determined using fluorescence by adding to the water a fluorophore compound, measuring a fluorescence signal of the water, and determining the amount of the sulfite in the water based on the measured fluorescence signal. This method can be used in a water treatment system in which a sulfite solution is added to treat the water, and the amount of sulfite that is added can be controlled based on the measured fluorescence of the water.

### CROSS-REFERENCE TO RELATED APPLICATION

This application claims the filing date benefit of U.S. Provisional Application No. 63/286,791, which was filed on Dec. 7, 2021. This application is incorporated by reference herein in its entirety.

### BACKGROUND

Sulfite is used in water treatment applications to scavenge and neutralize excess oxidizer in solution. For example, sulfite is used to scavenge excess dissolved oxygen in boilers to prevent corrosion. Sulfite is also used to neutralize bleach in wastewater applications so that excess bleach does not kill beneficial wastewater bacteria in downstream bioreactors. Sulfite is also used in reverse osmosis systems to neutralize bleach in feedwater to prevent breakdown of the polyamide structure of their membranes.

Sulfite is also used in food and beverages as a perseverative, antibacterial, and/or antioxidant agent. Since ingesting high levels of sulfite can cause health problems, various methods for detecting sulfite in food and beverage products have been developed, including e.g., electrochemistry, chromatography, titration, and flow injection analysis. Fluorescence detection has also been used to detect sulfites in food and beverage, but have been limited due to poor solubility, sensitivity, and/or detection limits. In some food and beverage applications, such as wine, sensitivity issues can be overcome by using high concentrations of the fluorophores, and solubility issues can be overcome due to the high alcohol content of the samples and/or by adding alcohol or other solvents to the samples.

However, there is no currently available method to quickly and economically measure sulfite residuals in water treatment applications such as in boilers, wastewater, reverse osmosis systems, municipal water, etc. Quantification of residual sulfite in these types of systems has been performed by methods such as ORP (Oxidation-Reduction Potential). However, ORP lacks the sensitivity needed to accurately control sulfite addition and there are frequent interferences that render the results unreliable. In these water treatment systems, fluorescence detection has not been used in water treatment systems due to the issues with solubility and sensitivity identified above. In this regard, the water that is being tested typically has lower level of sulfites than in food and beverage samples, the water has very low or no organic solvents, and, due to the volume of water and frequency of testing that is often needed, it is not feasible to add large amounts of solvents and/or large amounts of the fluorophore. Instead, in most industrial water treatment applications, sulfite is typically added in substantial excess of the expected amount that is needed, e.g., to scavenge oxygen and/or neutralize bleach.

### SUMMARY

Being able to reliably monitor sulfite residuals in water treatment applications is desirable to prevent overfeeding or underfeeding of sulfite.

In one aspect, this disclosure provides a water treatment system comprising (i) a sulfite container that contains a sulfite solution and is configured to supply the sulfite solution to water of the water treatment system at a first location, (ii) a reagent container that is configured to supply a fluorophore compound to the water at a second location downstream of the first location; and (iii) a fluorimeter that is configured to measure a fluorescence signal of the water at a third location that is downstream of the second location.

In another aspect, disclosure provides a method for determining the amount of sulfite in water which contains a concentration of sulfite that is in a range of from 0.1 ppm to 100 ppm, the method comprising (i) adding to the water a fluorophore compound at a concentration that is in a range of 1 ppb to 100 ppm; (ii) measuring a fluorescence signal of the water that includes the fluorophore compound; and (iii) determining the amount of the sulfite in the water based on the measured fluorescence signal.

### BRIEF DESCRIPTION OF THE DRAWINGS

FIG. 1 is a graph of a three-dimensional fluorescence scan showing the emission and excitation intensity of a coumarin-based fluorophore compound with no sulfite in water;

FIG. 2 is a graph of a three-dimensional fluorescence scan showing the emission and excitation intensity of a coumarin-based fluorophore compound with approximately 8 ppm sulfite in water;

FIG. 3 is a graph showing the fluorescence emission spectra of varying amounts of sulfite with a coumarin-based fluorophore;

FIG. 4 is a schematic diagram of a water treatment system according to one embodiment;

FIG. 5 is a schematic diagram of a water treatment system according to another embodiment;

FIG. 6 is a schematic diagram of inline fluorescence detection according to one embodiment; and

FIG. 7 is a schematic diagram of fluorescence detection using a split stream according to one embodiment.

### DETAILED DESCRIPTION OF EMBODIMENTS

In the following description, numerous details are set forth to provide an understanding of the present disclosure. However, it may be understood by those skilled in the art that the methods and systems of the present disclosure may be practiced without these details and that numerous variations or modifications from the described embodiments may be possible.

Disclosed herein are a methods and systems for determining the amount of residual sulfite in water, e.g., in a water treatment system, and for controlling the amount of sulfite that is added to the system based on the determined amount of residual sulfite. As used herein “water treatment system” means a water system in which sulfite is intentionally added to water to treat the water. The sulfite can be added to the system to treat the water for any reason including to scavenge and/or neutralize oxidizers such as oxygen or bleach that are present in the water. The water treatment system can include, for example, boiler water systems, reverse osmosis systems, municipal water systems, wastewater treatment systems, etc. The treated water stream to which the sulfite is added is at least 95 wt. % water, at least 99 wt. % water, or at least 99.5 wt. % water. In some aspects, the water can have less than 1 wt. % of organic solvents, such as alcohols, less than 0.5 wt. % of organic solvents, and in some cases can be free of organic solvents.

According to aspects of the invention, the amount of sulfite present in water can be determined and maintained at desired levels. For example, in boiler systems, sulfite levels in the boiler typically are kept above a minimum threshold level to ensure that dissolved oxygen in the boiler feedwater is rapidly and substantially removed. The sulfite is typically added to the water as a liquid solution of a sulfite salt, such as sodium sulfite. The amount of sulfite in the water that is analyzed can vary based on the application, but is generally in the range of about 0.1 ppm to 100 ppm. From 1 ppm to 50 ppm, and from 5 ppm to 25 ppm. As used herein, the weight of sulfite refers to the weight of the sulfite ion.

The methods described herein may include detecting the amount of sulfite in water of a water system by adding a suitable fluorophore to sulfite-containing water and inducing the fluorophore to fluoresce. Fluorescence of the fluorophore may be induced by applying an amount of energy to the water in the water system. The energy may be in the form of electromagnetic radiation, such as ultraviolet (UV) light, at a particular wavelength suitable for exciting the fluorophore. Electromagnetic radiation may also include infrared or visible light. The absorption of light by the fluorophore at a certain wavelength can be measured as the compound's excitation signal, or the emission of light at a certain wavelength after the compound has been exposed to an excitation wavelength can be measured as the compound's emission signal. The fluorescence signal can be measured at a wavelength that corresponds to the peak intensity of emission or absorption. As an example, the fluorophore can have a maximum excitation wavelength in a range of about 280 to 400 nm, or 320 nm to 375 nm, and can have a maximum emission wavelength in a range of about 400 to 600 nm, or 425 nm to 500 nm.

The fluorophore is selected so that it interacts or reacts with the sulfite in the water, and so that the fluorescence signal intensity changes based on the amount of sulfite that is dissolved in the water, e.g., the fluorescence emission intensity is inversely proportional or directly proportional to the amount of sulfite. This allows for a direct correlation of fluorescence signal intensity to sulfite concentration.

A standard curve can be determined from the relationship between the intensity of the fluorescence signal and the concentration of the sulfite so that the amount of the residual sulfite in the water treatment system can be quantified. For example, to determine the standard curve, the fluorescence signal of water with the fluorophore is measured in the presence of various known concentrations of sulfite. The fluorescence signal is typically measured at the wavelengths at which the fluorophore compound exhibits peak excitation and/or emission. The intensity of the signals are plotted against the concentration of the sulfite, and a regression of these data points is performed (e.g., linear regression). The concentration of sulfite in the assayed water can be determined by comparing the signal intensity to the standard curve.

As described above, in water treatment systems it often is not practical to add large amounts of fluorophore compound to the water, e.g., due to the volume of water that needs to be assayed. Accordingly, in one aspect, the fluorophore compound can be sufficiently sensitive that it can provide a signal intensity that allows for reliable quantification of the sulfite even where the fluorophore compound is added in low concentrations. For example, the fluorophore compound can be sufficiently sensitive that it is used in the water at concentrations of less than 100 ppm, in the range of from 1 ppb to 10 ppm, from 10 ppb to 1 ppm, from 50 ppb to 0.5 ppm, or from 75 ppb to 250 ppb. The fluorophore compound can be added in amounts of 0.25 wt. % to 25 wt. % based on the weight of the sulfite that is being detected in the water, from 0.5 wt. % to 10 wt. % based on the weight of the sulfite, or from 1 wt. % to 5 wt. % based on the weight of sulfite.

In another aspect, the fluorophore compound can be soluble in pure water at neutral pH and standard conditions at the aforementioned concentrations (i.e., such that at least 95 wt. % of the fluorophore compound dissolves). In some aspects, the fluorophore compound can be synthesized by reacting (i) a compound with a moiety that reacts with the sulfite in solution (e.g., levulinate); and (ii) a solubility-enhancing compound (e.g., coumarin), such that the reaction product can detect sulfite and has improved solubility. In some aspects, one or both of the moieties that reacts with the sulfite and the solubility-enhancing compound can include a fluorophore.

In some aspects, the fluorophore compound can include one or more of a coumarin moiety, fluorescein moiety, and an anthracene moiety. In some aspects, any of these moieties can be linked with at least one of an ester moiety or an aldehyde moiety. In one example, the fluorophore compound can be a reaction product of levulinic acid and coumarin, e.g., based on the reaction shown below.

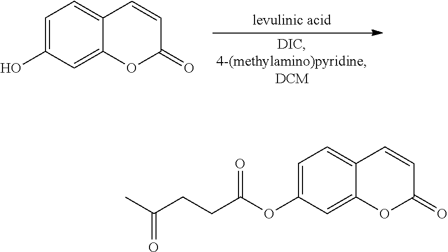 [SMILES: O=c1ccc2ccc(O)cc2o1 ; CC(=O)CCC(=O)Oc1ccc2ccc(=O)oc2c1]

This reaction produces a fluorophore compound that provides a good signal even at relatively low concentrations and is sensitive to sulfite even in the presence of other ions. In this regard, it is believed that the sulfite interacts with the carbonyl of the levulinate moiety which causes the cleavage of the ester bond to form hydroxycoumarin.

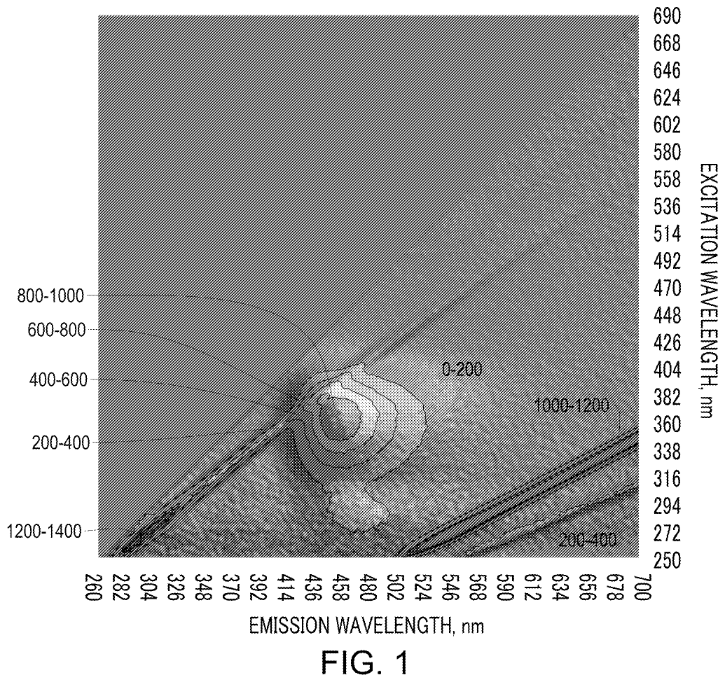

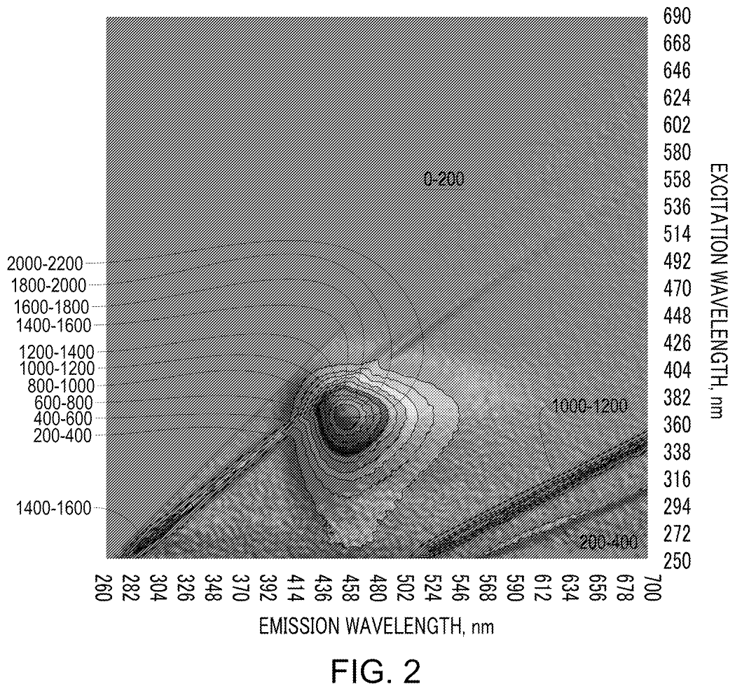

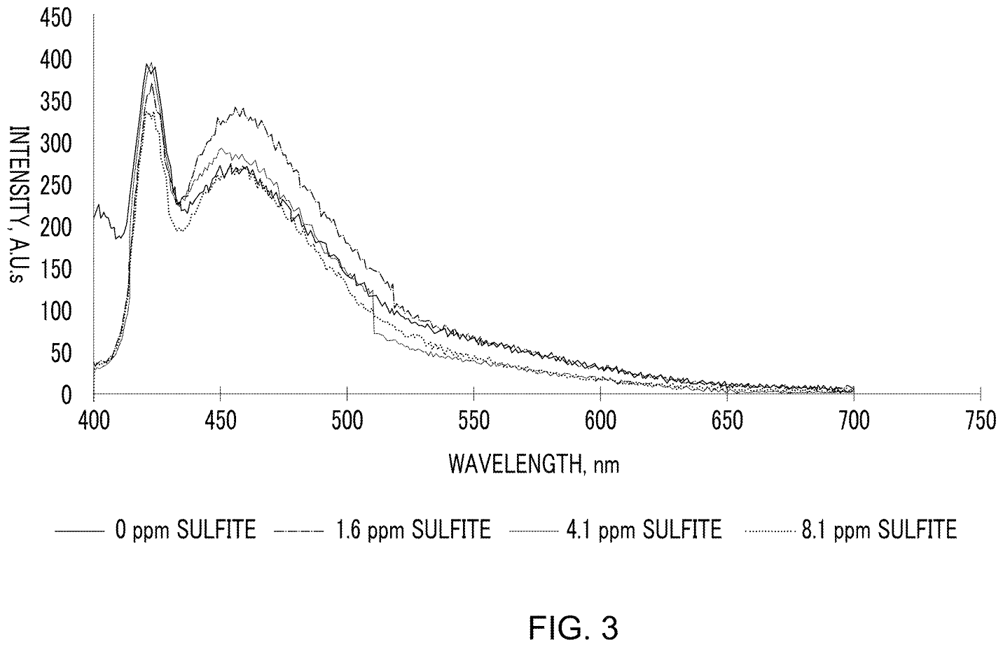

FIG. 1 is a three-dimensional fluorescence scan showing an excitation peak of 364 nm and an emission peak at 456 nm (984 A.U.) of the levulinate-coumarin fluorophore compound with no sulfite in water. FIG. 2 is a three-dimensional fluorescence scan showing an excitation peak of 368 nm and an emission peak at 456 nm (2210 A.U.) of the levulinate-coumarin fluorophore compound and about 8 ppm sulfite in water.

Embodiments of the disclosed methods allow for the real-time detection and quantification of the residual sulfite in the water. Detection and quantification of the sulfite can therefore be achieved more quickly, at a lower cost, and without the need for sophisticated equipment and training. This allows for greater control of the quantity of sulfite that is added to the water system, both to ensure that sufficient sulfite is present and to ensure that too much sulfite is not added to the system, for example, for cost reasons and/or to prevent excess sulfite from being present in the waste stream.

The amount of sulfite can be controlled by adding a suitable fluorophore to the sulfite-containing water, causing the fluorophore to fluoresce, and measuring an intensity of the fluorescence signal from the water to determine the concentration of the sulfite in the water by any of the techniques discussed above. The method can include adjusting the amount of sulfite that is added to the water based on the determined concentration of residual sulfite. For example, the determined amount of sulfite can be compared to a predetermined threshold value, and if the amount of sulfite exceeds the threshold value, the amount of sulfite being added to the water can be reduced. Likewise, if the determined amount of sulfite is below a certain value, the amount of sulfite that is being added to the water can be increased. The amount of sulfite can be automatically and/or continuously, intermittently, or periodically controlled by a controller, such as a CPU, that adjusts the amount of sulfite that is added to the water based on one or more feedback loop mechanisms (e.g., PID controller) based on the fluorescence readings.

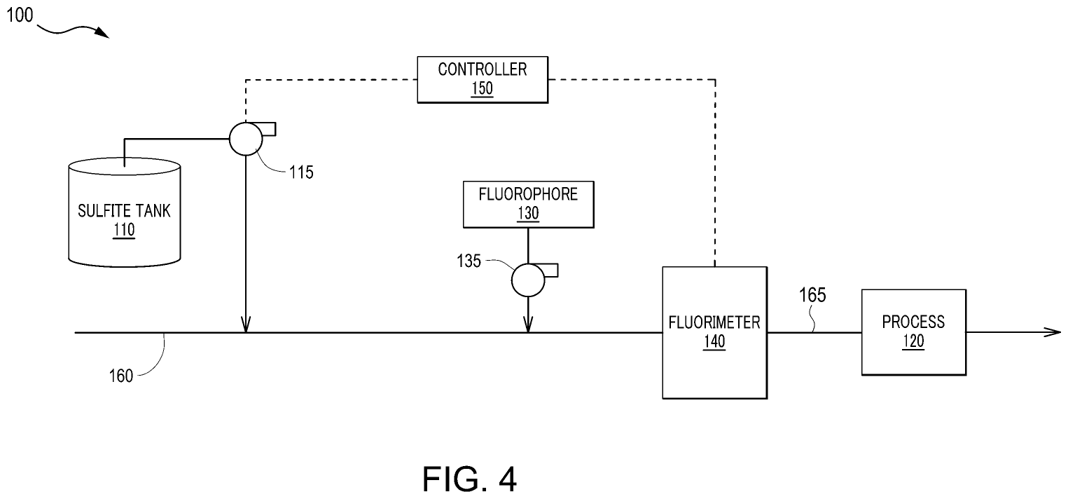

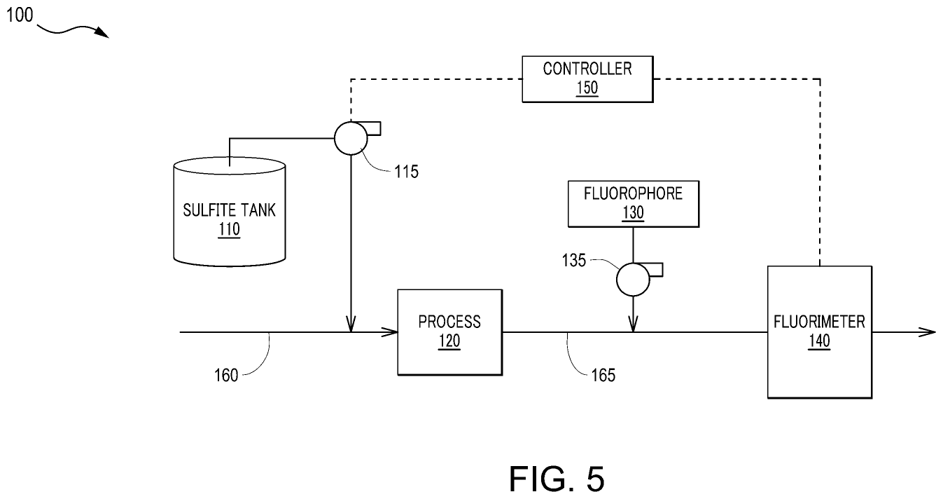

FIGS. 4 and 5 illustrate embodiments of a water treatment system 100 in which the amount of residual sulfite that is present in the water can be detected, and the amount of sulfite that is added to the water can be controlled. The system 100 includes a sulfite tank 110 (or other container) with pump 115, a process 120, a water stream 160 upstream of process 120, and a water stream 165 downstream of process 120, a fluorophore reservoir 130 with pump 135, a fluorimeter 140, and a controller 150.

The process 120 can be any process that is part of the water treatment system. For example, process 120 can be a process that uses a boiler, heat exchanger, filter, reverse osmosis membrane, bioreactor, etc. In the FIG. 4 embodiment, the process 120 is downstream of the fluorimeter 140. This arrangement may be preferred in situations where it is important to ensure that a minimum amount of sulfite is present in the process 120, such as in a boiler. In such cases, the control of the sulfite addition is more responsive if the sulfite is detected upstream of the process 120, particularly if the water in the process 120 has a high residence time. In the FIG. 5 embodiment, the process 120 is located upstream of the water. This arrangement may be useful where the sulfite is consumed in the process 120 and it is important to ensure that a sufficient amount of sulfite is added to meet the demands of the process, e.g., so that a minimum threshold amount of sulfite is present in the process effluent. In other embodiments, the sulfite can be detected both upstream and downstream of process 120. In still other embodiments, there is no such process 120.

The water treatment system 100 includes a sulfite tank 110 that contains a liquid solution of sulfite. The sulfite solution can be pumped into stream 160 of the water treatment system 100 via pump 115.

Fluorophore reservoir 130 is a container that includes a suitable fluorophore compound that can be added to stream 160 (FIG. 4) or 165 (FIG. 5) via pump 135. In some aspects, pump 135 can be replaced with a volumetric injector that injects known quantities of the fluorophore into the stream. The fluorophore compound can react with the sulfite present in the water upstream of the fluorimeter 140. The fluorophore reservoir 130 can be placed sufficiently upstream to allow the fluorophore compound to substantially react with the water prior to reaching the fluorimeter. Thus, the fluorophore reservoir 130 and fluorimeter 140 can be positioned at a relative distance based on the flow rate of the water and the reaction time of the fluorophore compound with sulfite. For example, these components can be placed at a distance that allows the fluorophore compound to react with the sulfite for a time period in the range of from 1 second to 2 minutes, from 5 seconds to 1 minute, and from 10 seconds to 45 seconds prior to reaching the fluorimeter 140.

The fluorimeter 140 detects the fluorescence signal of the fluorophore compound in the water and transmits information relating to the fluorescence signal to controller 150. The controller 150 can determine the amount of sulfite in the water, e.g., by comparing the detected fluorescence signal to a standard curve as discussed above. The controller 150 can also generate signals that control the amount of sulfite added to the water from sulfite tank 110 based on the detected amount of sulfite in the water. For example, the controller can compare the detected amount of sulfite to a predetermined threshold, and increase the amount of sulfite added to the water if the detected amount of sulfite is below a predetermined threshold, and likewise can decrease the amount of sulfite that is added to the water if the detected amount is above a predetermined threshold. Likewise, the controller can compare the detected amount of sulfite to a look up table that identifies the amount of sulfite that should be added based on the amount of sulfite that is detected. The controller 150 can send the control signals so that pump 115 adjusts the amount of sulfite that is added to the water.

The controller can include hardware, such as a circuit for processing digital signals and/or a circuit for processing analog signals, for example. The controller may include one or a plurality of circuit devices (e.g., an IC) or one or a plurality of circuit elements (e.g., a resistor, a capacitor) on a circuit board, for example. The controller may be a central processing unit (CPU) or any other suitable processor. The controller may be or form part of a specialized or general purpose computer or processing system. One or more controllers, processors, or processing units, memory, and a bus that operatively couples various components, including the memory to the controller, may be used. The controller may include a module that performs the methods described herein. The module may be programmed into the integrated circuits of the processor, or loaded from memory, storage device, or network or combinations thereof.

The controller may be operational with numerous other general purpose or special purpose computing system environments or configurations. Examples of well-known computing systems, environments, and/or configurations that may be suitable for use with the disclosed embodiments may include, but are not limited to, personal computer systems, server computer systems, thin clients, thick clients, handheld devices, such as tablets and mobile devices, laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

The various components of the water treatment system may be connected with each other via any type of digital data communication such as a communication network. Data may also be provided to the process controller through a network device, such as a wired or wireless Ethernet card, a wireless network adapter, or any other device designed to facilitate communication with other devices through a network. The network may be, for example, a Local Area Network (LAN), Wide Area Network (WAN), and computers and networks which form the Internet. The system may exchange data and communicate with other systems through the network. For example, the method may be practiced in clouding computing environments, including public, private, and hybrid clouds. The method can also or alternatively be practiced in distributed cloud computing environments where tasks are performed by remote processing devices that are linked through a communications network. In a distributed cloud computing environment, program modules may be located in both local and remote computer system storage media including memory storage devices. The system may be also be configured to work offline.

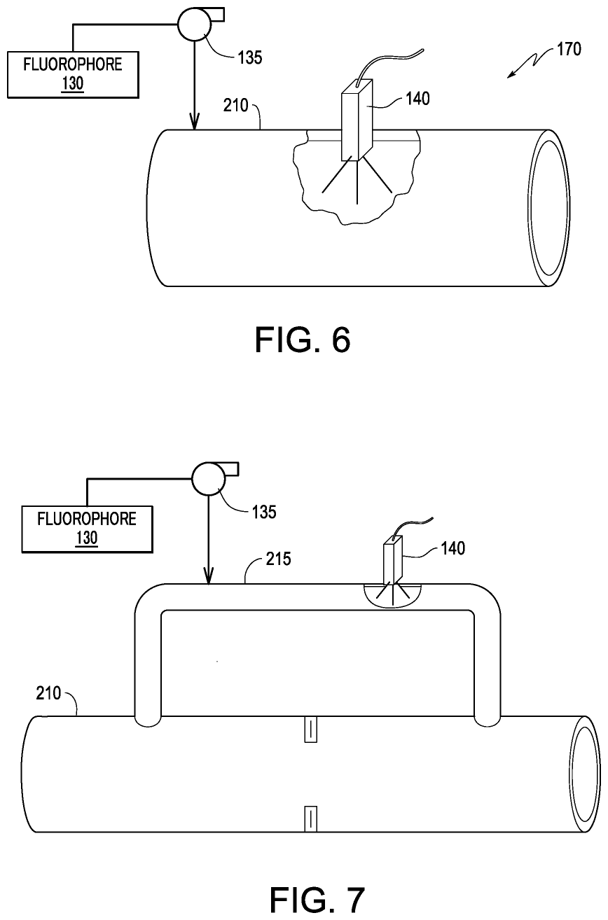

FIGS. 6 and 7 show additional details of an in-line fluorescence detection unit that can include the fluorophore reservoir 130 and the fluorimeter 140. For example, as shown in FIG. 6, a conduit 210 of the water treatment system 100 can carry the sulfite-containing water. Fluorophore compound can be added via pump 135 to a conduit 210 upstream of the fluorimeter 140 so that the fluorophore compound sufficiently reacts with the sulfite so that the change in fluorescence signal caused by the sulfite can be measured by the fluorimeter 140. The fluorimeter 140 in this embodiment is mounted to conduit 210, and sends the detected fluorescence signals to the controller 150. Alternatively, as shown in FIG. 7, the fluorescence detector can be mounted on a slipstream 215 of conduit 210 where the fluorophore is added to the slip stream upstream of the fluorimeter 140.

It will be appreciated that the above-disclosed features and functions, or alternatives thereof, may be desirably combined into different systems or methods. Also, various alternatives, modifications, variations or improvements may be subsequently made by those skilled in the art. As such, various changes may be made without departing from the spirit and scope of this disclosure.

What is claimed is:

1. A water treatment system comprising:
a sulfite container that contains a sulfite solution and is configured to supply the sulfite solution to water of the water treatment system at a first location,
a reagent container that is configured to supply a fluorophore compound to the water at a second location downstream of the first location; and
a fluorimeter that is configured to measure a fluorescence signal of the water at a third location that is downstream of the second location.

2. The water treatment system according to claim 1, wherein the water treatment system further includes at least one controller that is configured to receive information regarding the fluorescence signal measured by the fluorimeter and determine an amount of sulfite in the water based on the fluorescence signal.

3. The water treatment system according to claim 2, wherein the at least one controller is configured to send a signal to control the amount of the sulfite solution that is supplied to the water based on the determined amount of sulfite in the water.

4. The water treatment system according to claim 1, wherein the water treatment system includes a boiler that is downstream of the sulfite container so that sulfite-containing water is supplied as feedwater to the boiler.

5. The water treatment system according to claim 4, wherein the boiler is downstream of the fluorimeter.

6. The water treatment system according to claim 1, wherein the water to which the sulfite is added includes bleach.

7. The water treatment system of claim 6, further comprising a membrane that is located downstream of the first location through which the water passes.

8. The water treatment system of claim 6, further comprising a bioreactor that is located downstream of the second location.

9. The water treatment system according to claim 1, wherein the fluorophore compound includes a coumarin moiety.

10. The water treatment system according to claim 1, wherein the fluorophore compound includes an ester moiety.

11. A method for determining the amount of sulfite in water which contains a concentration of sulfite that is in a range of from 0.1 ppm to 100 ppm, the method comprising:
(i) adding to the water a fluorophore compound at a concentration that is in a range of 1 ppb to 100 ppm;
(ii) measuring a fluorescence signal of the water that includes the fluorophore compound and the sulfite; and
(iii) determining the amount of sulfite in the water based on the measured fluorescence signal.

12. The method of claim 11, wherein the fluorophore compound is a reaction product of (i) a compound including at least one of a coumarin moiety, a fluorescein moiety, and an anthracene moiety; and (ii) a compound with a moiety that reacts with sulfite.

13. The method of claim 11, wherein the compound with a moiety that reacts with sulfite is levulinic acid.

14. The method of claim 11, wherein the fluorophore compound includes a coumarin moiety and an ester moiety.

15. The method of claim 11, wherein the fluorophore compound is added to the water at a concentration that is in a range of from 10 ppb to 10 ppm.

16. The method of claim 11, wherein the sulfite is present in the water at a concentration in the range of from 1 ppm to 50 ppm.
